(19) United States
(12) Patent Application Publication
(10) Pub. No.: US 2007/0123498 A1
(43) Pub. Date: May 31, 2007

(54) Combination of organic compounds

(76) Inventors: Suraj Shivappa Shetty, Andover, MA (US); Gary Michael Ksander, Amherst, NH (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/574,745

(22) PCT Filed: Oct. 15, 2004
(86) PCT No.: PCT/EP04/11652

§ 371 Date: Apr. 6, 2006

Related U.S. Application Data
(60) Provisional application No. 60/512,515, filed on Oct. 17, 2003.

(51) Int. Cl. A61K 31/58; A61K 31/549; A61K 31/522; A61K 31/4184

(52) U.S. Cl. 514/171; 514/223.5; 514/176; 514/263.31; 514/394

(57) ABSTRACT

The present invention relates to a combination of organic compounds, a pharmaceutical composition and a kit of parts comprising said combination of organic compounds and to a method of treatment or prevention of certain conditions or diseases.

The present invention relates to a combination of organic compounds that are antihypertensive agents with complementary modes of action for eliciting blood pressure-lowering, and also for attenuating the varied pathological sequelae of hypertension and several other cardiovascular disorders. Furthermore, this invention addresses the disparate responsiveness of humans to antihypertensive monotherapy, based on age and/or ethnicity (Campo C, Segura J, Ruilope L M, J Clin Hypertens (Greenwich) 2002 January, 4(1):35-40). Finally, the choice of agents and their respective dosages in the combination regirnen are designed to enhance tolerability by minimizing the risk of dose-dependent adverse effects associated with individual agents.

Numerous clinical studies have shown that lowering blood pressure in hypertensive patients reduces mortality and morbidity (Collins R, Peto R, MacMahon S, Hebert P, Fiebach N H, Eberlein K A, Godwin J, Qizilbash N, Taylor J O, Hennekens C H, Lancet 1990, 335(8693):827-38). Despite the availability and use of various classes of agents in the treatment of this medical condition, adequate control of blood pressure is not always achieved (Waeber B, Brunner H R, Am J Hypertens 1997, 10(7 Pt 2):131S-137S). Using a combination of agents is one way to achieve the desired therapeutic end-point. An arbitrary selection of antihypertensive agents of different classes for inclusion in a combination therapy regimen does not necessarily help achieve target levels of blood pressure in hypertensive mammals including humans (MacGregor G A, Markandu N D, Banks R A, Bayliss J, Roulston J E, Jones J C, Br Med J (Clin Res Ed) 1982, 284(6317):693-6). Therefore, a need for further development of methods of treatment, combinations, and pharmaceutical compositions clearly exists.

Specifically, the present invention relates to a combination comprising (i) a mineralocorticoid receptor antagonist (also referred to as aldosterone receptor antagonist or aldosterone antagonist) or pharmaceutically acceptable salts thereof; (ii) a diuretic or pharmaceutically acceptable salts thereof, and optionally (iii) an angiotensin receptor (Type 1, AT₁) blocker (ARB) or pharmaceutically acceptable salts thereof; optionally in the presence of a pharmaceutically acceptable carrier. The invention further provides methods for treating hypertension and a variety of cardiovascular disorders enumerated below and their sequelae by administration of the pharmaceutical composition comprising (i) a mineralocorticoid receptor antagonist or pharmaceutically acceptable salts thereof; (ii) a diuretic or pharmaceutically acceptable salts thereof, and optionally (iii) an angiotensin receptor (Type 1, AT₁) blocker (ARB) or pharmaceutically acceptable salts thereof; to a mammal including humans.

Preferably the present invention relates to a combination comprising (i), (ii) and (iii).

A combination according to the present invention can be in the form of a combined preparation or a pharmaceutical composition.

Angiotensin receptor (Type 1, AT₁) blockers (also called angiotensin II receptor antagonists) are understood to be those active ingredients which bind to the AT₁-receptor subtype of angiotensin II receptor but do not result in activation of the receptor. As a consequence of the inhibition of the AT₁ receptor, these antagonists can, for example, be employed as antihypertensives or for treating congestive heart failure.

The class of AT₁ receptor blockers comprises compounds having differing structural features, essentially preferred are the non-peptidic ones. For example, mention may be made of the compounds which are selected from the group consisting of valsartan, losartan, candesartan, eprosartan, irbesartan, saprisartan, tasosartan, telmisartan, olmesartan, the compound with the designation E-4177 of the following formula 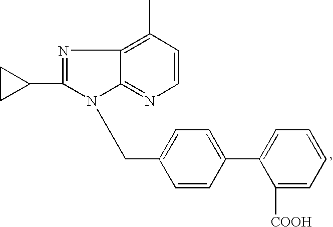
the compound with the designation SC-52458 of the following formula 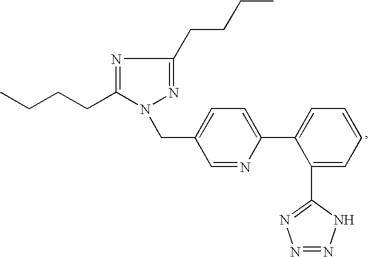
and the compound with the designation ZD-8731 of the following formula 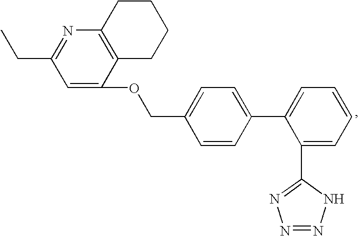
or, in each case, a pharmaceutically acceptable salt thereof.

Preferred AT₁-receptor blockers are selected from the group consisting of candesartan, eprosartan, irbesartan, losartan, olmesartan, saprisartan, tasosartan, telmisartan, valsartan, E-4177, SC-52458, and ZD8731, or pharmaceutically acceptable salts thereof

Preferred AT₁-receptor blockers are those agents which have been marketed, most preferred is valsartan or a pharmaceutically acceptable salt thereof.

Mineralocorticoid receptor antagonists (or aldosterone receptor antagonists) are compounds capable of inhibiting the binding of aldosterone to its receptor, and thus modulating the receptor- mediated activity of aldosterone.

The aldosterone antagonists of the present invention are spirolactone-type steroidal compounds.

The term “spirolactone-type” is intended to characterize a structure which comprises a lactone moiety attached to a steroid nucleus, preferably at the steroid “D” ring, through a spiro bond configuration.

A subclass of spirolactone-type aldosterone antagonist compounds consists of epoxy-steroidal aldosterone antagonist compounds such as eplerenone. Examples of preferred epoxy-steroidal aldosterone antagonists are described in the US patent US2003/0149010 published on Aug. 7, 2003, in table I and are incorporated herein by reference.

Another subclass of spirolactone-type antagonist compounds consists of non-epoxy-steroidal aldosterone antagonist compounds such as spironolactone. Examples of preferred non-epoxy-steroidal aldosterone antagonists are described in the US patent US2003/0149010 published on Aug. 7, 2003, on pages 7 to 9 and are incorporated herein by reference.

By the term “epoxy-steroidal aldosterone receptor antagonist” is intended to embrace one or more agents or compounds characterized by a steroid-type nucleus (i.e. provided by a cyclopentenophenanthrene moiety) and having one, two or more epoxy moiety attached to the nucleus and which agent or compound binds to the aldosterone receptor, as a competitive inhibitor of the action of aldosterone itself at the receptor site, so as to modulate the receptor-mediated activity of aldosterone. The term “epoxy-type” moiety is intended to embrace any moiety characterized in having an oxygen atom as a bridge between two carbon atoms, such as an epoxyethyl, an 1,3-epoxypropyl or an 1,2-epoxypropyl. The epoxy-type moiety may be attached to the cyclopentenophenanthrene nucleus (i.e. having the conventional “A”, “B”, “C” and “D” rings) at any attachable or substitutable positions, that is, fused to one of the rings of the steroidal nucleus or the moiety may be substituted on a ring member of the ring system.

A preferred epoxy-steroidal aldosterone receptor antagonist is a spirolactone-type aldosterone known by the common name epoxymexrenone and also by the USAN designation eplerenone (see European patent EP 122232 A) of the formula

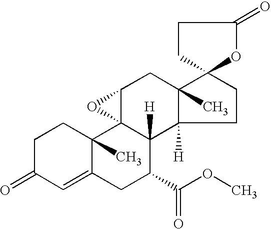

Preferred non-epoxy-steroidal aldosterone antagonist is spironolactone. Spironolactone is the name commonly used by chemists; the full chemical name is 17-hydroxy-7-alpha-mercapto-3-oxo-17-alpha-pregn-4-ene-21-carboxylic acid gamma-lactone acetate. This compound, its activities, and modes of synthesis and purification are described in a number of U.S. patents, including U.S. Pat. No. 3,013,012 (Cella and Tweit 1961) and U.S. Pat. No. 4,529,811 (Hill and Erickson 1985).

The compound methyl hydrogen 9,11-epoxy-17-hydroxy-3-oxopregn-4-ene-7,21-dicarboxylate, γ-lactone known as eplerenone was first reported in U.S. Pat. No. 4,559,332 to Grob et al., which discloses a class of 9,11-epoxy steroid compounds and their salts. Above-cited U.S. Pat. No. 4,559,332, which is incorporated herein by reference, generally discloses preparation of eplerenone and preparation of pharmaceutical compositions comprising eplerenone.

Eplerenone or spironolactone refer to all kind of crystalline or amorphous forms such as solvated crystalline eplerenone, non-solvated crystalline eplerenone, and amorphous eplerenone. The preferred crystalline forms of eplerenone are selected from the Form H and Form L such as described in the US patent applications US20030055027 or US20020045746 especially by the examples and claims, which are incorporated herewith by reference.

The term “amorphous” as applied to eplerenone herein refers to a solid state wherein the eplerenone molecules are present in a disordered arrangement and do not form a distinguishable crystal lattice or unit cell. When subjected to X-ray powder diffraction, amorphous eplerenone does not produce any characteristic crystalline peaks.

The term “crystalline form” as applied to eplerenone herein refers to a solid state form wherein the eplerenone molecules are arranged to form a distinguishable crystal lattice (i) comprising distinguishable unit cells, and (ii) yielding diffraction peaks when subjected to X-ray radiation.

The diuretic that, according to the present invention, is used in combination with the angiotensin (AT1) receptor blocker and the aldosterone antagonists is preferably selected from the group consisting of bumetanide, ethacrynic acid, furosemide, mercaptomerin sodium, torsemide, amiloride, triamterene, chlorthalidone, chlorothiazide, quinethazone, hydrochlorothiazide (HCTZ), hydroflumethiazide, methylchlorothiazide, metolazone, and dichlorphenamide.

A preferred diuretic is, for example, a thiazide derivative selected from the group consisting of chlorothiazide, hydrochlorothiazide, and chlorthalidone.

The most preferred diuretic for the intended combination is a thiazide diuretic, e.g. hydrochlorothiazide.

The active agents mentioned herein cover all kind of pharmaceutically acceptable salt thereof; diastereomer thereof; mixture of diastereomers thereof; optical isomer thereof; mixture of optical isomers thereof, crystalline or amorphous forms thereof.

The structure of the active agents identified by generic or tradenames may be taken from the actual edition of the standard compendium “The Merck Index” or from databases, e.g. LifeCycle Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

Thus, the invention further relates to a combination, or a kit of parts, e.g. for the treatment or prevention of a condition or disease selected from the group consisting of hypertension, all types of heart failures including acute and chronic congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina pectoris (whether unstable or stable), renal insufficiency (diabetic and non-diabetic), diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke which combination, or kit of parts, comprises comprising (i) an aldosterone antagonist; (ii) a diuretic, and optionally (iii) an angiotensin receptor (Type 1, AT₁) blocker or, where appropriate, a pharmaceutically acceptable salt thereof and a pharmaceutically acceptable carrier.

Preferably the kit of parts comprises (i), (ii) and optionally (iii).

A further aspect of the present invention is a method for the treatment or prevention of a condition or disease selected from the group consisting of hypertension, all types of heart failures including acute and chronic congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke, comprising administering a therapeutically effective amount of combination of (i) an aldosterone antagonist; (ii) a diuretic, and optionally (iii) an angiotensin receptor (Type 1, AT₁) blocker (ARB) or, where appropriate, a pharmaceutically acceptable salt thereof and a pharmaceutically acceptable carrier, to a mammal in need of such treatment.

Preferably the invention concerns a method comprising administering a therapeutically effective amount of combination of (i), (ii) and optionally (iii).

A combination according to the present invention can be in the form of a combined preparation or a pharmaceutical composition (fixed combination).

The invention also relates to combining separate pharmaceutical compositions in kit form. That is a kit combining two or three separate units: e.g. a pharmaceutical composition comprising an ARB, a pharmaceutical composition comprising a diuretic and a pharmaceutical composition comprising an aldosterone antagonist; or a pharmaceutical composition comprising an ARB and a diuretic and a pharmaceutical composition comprising an aldosterone antagonist; or a pharmaceutical composition comprising an aldosterone antagonist and a diuretic, and a pharmaceutical composition comprising an ARB, or a pharmaceutical composition comprising an ARB and an aldosterone antagonist, and a pharmaceutical composition comprising a diuretic. Although the kit form is particularly advantageous when the separate components must be administered in different dosage forms (e.g. parenteral valsartan formulation and oral eplerenone or hydrochlorothiazide formulations) or are administered at different dosage intervals, the administration of the single components of such a kit of parts may, without any restriction be effected simultaneously, sequentially or staggered with time.

In a preferred embodiment, the (commercial) product is a commercial package comprising as active ingredients the combination according to the present invention (in the form of two or three separate units of the components (i) to (ii) or (i) to (iii)), together with instructions for its simultaneous, separate or sequential use, or any combination thereof, in the delay of progression or treatment of the diseases mentioned herein. A preferred commercial package, is where the ARB (iii) and the diuretic (ii) are present in the form of Co-DIOVAN®, or where the aldosterone antagonist (i), the ARB (iii) and the diuretic (ii) are present in the form of Co-DIOVAN® and INSPRA®, or where the aldosterone antagonist (i) and the ARB (iii) are present in the form of DIOVAN®, INSPRA®. In a further embodiment the ARB (iii) is not present.

The pharmaceutical preparations of the present invention are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compounds. Pharmaceutical preparations for enteral or parenteral administration are, for example, in unit dose forms, such as coated tablets, tablets, capsules or suppositories and also ampoules. These are prepared in a manner, which is known per se, for example using conventional mixing, granulation, coating, solubilizing or lyophilizing processes. Thus, pharmaceutical preparations for oral use can be obtained by combining the active compounds with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition. Preferred dosages for the active ingredients of the pharmaceutical combination according to the present invention are therapeutically effective dosages, especially those that are commercially available. Normally, in the case of oral administration, an approximate daily dose of from about 5 mg to about 1000 mg of active agents, i.e. ARB plus aldosterone antagonist plus diuretic, is to be estimated e.g. for a patient of approximately 75 kg in weight.

In the present invention preferred ARBs are those agents that have been marketed, as e.g. valsartan and losartan. The preferred aldosterone antagonist employed in the present invention, are eplerenone and spironolactone. The most preferred diuretic is hydrochlorothiazide (HCTZ).

All the more surprising is the experimental finding that the combined administration of (i) an aldosterone antagonist, (ii) a diuretic, and optionally (iii) an ARB, or independently of each other a salt thereof, results not only in a beneficial, especially a synergistic, therapeutic effect, but also in additional benefits resulting from the combined treatment and further surprising beneficial effects compared to a monotherapy applying only one of the pharmaceutically active compounds used in the combinations disclosed herein.

Very surprisingly is the finding that, a combination of (i) an aldosterone antagonist, (ii) a diuretic, and optionally (iii) an ARB, preferably a combination of (i) an aldosterone antagonist, (ii) a diuretic, and (iii) an ARB, and in particular a combination comprising HCTZ, eplerenone and optionally valsartan achieves greater therapeutic effect than the administration of valsartan, eplerenone, or HCTZ alone or in a combination of two of these agents. Greater efficacy can also be documented as a prolonged duration of action. The duration of action can be monitored as either the time to return to baseline prior to the next dose or as the area under the curve (AUC) and is expressed as the product of the change in blood pressure in millimeters of mercury (change in mmHg) and the duration of the effect (minutes, hours or days). The aforementioned combination treatment also unexpectedly reduces blood pressure in hypertensive mammals in a smooth and sustained fashion. The trough:peak blood pressure ratio demonstrated by this combination is close to unity leading to a more homogenous blood pressure control during the inter-dosing period. The combined regimen is almost completely devoid of either orthostatic hypotension or first-dose hypotension, and incidences of rebound hypertension after cessation of treatment are very rare. It can be shown that combination therapy according to the invention results in lessening of pulse pressure in hypertensive mammals.

Furthermore, this combination therapy can ameliorate endothelial dysfunction and improve vascular compliance and distensibility in hypertensive mammals. It can also slow the progression of cardiac, renal and cerebral end-organ damage in these mammals. Further benefits are that lower doses of the individual drugs to be combined according to the present invention can be used to reduce the dosage, for example, that the dosages need not only often be smaller but are also applied less frequently, or can be used to diminish the incidence of side effects.

In particular the combined administration of eplerenone or a pharmaceutically acceptable salt thereof, HCTZ and optionally valsartan or a pharmaceutically acceptable salt thereof, results in a significant response in a greater percentage of treated patients compared to monotherapy or combination therapy e.g. valsartan and HCTZ, that is, a greater responder rate results, regardless of the underlying etiology of the condition. This is in accordance with the desires and requirements of the patients to be treated. The combination treatment effectively lowers blood pressure in hypertensive patients in all age groups including pre and postmenopausal women. It can be shown that combination therapy with eplerenone, HCTZ and optionally valsartan, results in a more effective antihypertensive therapy (whether for malignant, essential, reno-vascular, diabetic, isolated systolic, or other secondary type of hypertension) and lessening of pulse pressure through improved efficacy. The combination is also useful in the treatment or prevention of heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter or detrimental vascular remodeling. It can further be shown that eplerenone, HCTZ and optionally valsartan combination therapy proves to be beneficial in the treatment and prevention of myocardial infarction and its sequelae. An eplerenone, HCTZ and optionally valsartan combination is also useful in treating atherosclerosis, angina (whether stable or unstable), renal insufficiency (diabetic and non-diabetic), peripheral vascular disease, cognitive dysfunction, and stroke. Furthermore, the improvement in endothelial function with the combination therapy using eplerenone, HCTZ and optionally valsartan provides benefit in diseases in which normal endothelial function is disrupted such as heart failure, angina pectoris and diabetes. Furthermore, the combination of the present invention may be used for the treatment or prevention of secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke. The combination regimen also surprisingly reduces the rate of progression of cardiac, renal and cerebral end-organ damage. By providing enhanced efficacy, safety and tolerability, the combination of drugs indicated in this invention also has the potential to promote patient compliance, a major consideration In the pharmacological treatment of hypertension.

The person skilled in the pertinent art is fully enabled to select a relevant test model to prove the efficacy of a combination of the present invention in the herein before and hereinafter indicated therapeutic indications.

The advantages of the present combinations are, for example, demonstrated in a clinical study or in the test procedure as essentially described hereinafter. Many clinical study protocols adapted to test our combinations are known by the person skilled in the art. An example of a clinical trial useful to demonstrate the unexpected advantages of our new combinations is described by Waeber B, Aschwanden R, Sadecky L, Ferber P (J Hypertens. 2001 November ; 19(11):2097-104. Similar protocol is performed with our preferred combinations such as a combination of hydrochlorothiazide 12.5 mg, eplerenone 25 or 50 mg and optionally valsartan 80 mg. The combination may a fixed-dose combination or not. This protocol is hereby incorporated into the present application by reference to this publication.

Representative studies are carried out with a combination of valsartan, eplerenone, and HCTZ applying the following methodology. Drug efficacy is assessed in various animal models including the stroke-prone spontaneously hypertensive rats (SHRsp) and the spontaneously hypertensive/heart failure rats (SHHF).

Blood Pressure and Heart Rate Effects in SHRsp:

The SHRsp exhibits accelerated hypertension and target organ damage when subjected to salt supplementation.

Methods: Surgery for implantation of a radiotransmitter is performed in the animals at 10 weeks of age. Radiotransmitters are implanted as described previously (Webb R L, Navarrete A E, Davis S, Am J Hypertens. 1998; 11(1 Pt 1):59-65). After a one-week recovery period, the SHRsp are placed on salt-supplemented drinking water (1% NaCl solution) and standard rodent chow. Drug treatments (monotherapy or combination therapies) are also initiated at this time and continued for the next 10 weeks.

Blood Pressure and Heart Rate Effects in SHHF

The SHHF is an animal model of spontaneous hypertension and congestive heart failure. These animals exhibit symptoms and pathophysiological changes that are observed in patients with hypertension, cardiomyopathy and congestive heart failure.

Methods: Male SHHF are used at 13 months of age. All animals are implanted with radiotransmitters according to the procedures described previously (Webb R L, Navarrete A E, Davis S, Am J Hypertens. 1998;11(1 Pt 1):59-65). The animals are allocated to the various treatment groups based on baseline blood pressure measurements. Oral dosing is commenced in the animals one week later and continued for the next 12 weeks.

Protocols for both the experimental models are set up on a computer for measurement of blood pressure, heart rate, etc, at predetermined time points. Baseline data is collected at various time points and over various time intervals. For example, baseline or pre-dose values usually consist of data collection and averaging over 3 consecutive, 24-hour time periods prior to drug administration. Blood pressure, heart rate and activity are determined at various pre-selected time points before, during, and after drug administrations. All measurements are performed in unrestrained and undisturbed animals. For chronic studies, rats are either orally dosed once daily or the drugs are administered via the drinking water or as admixtures in food.

Valsartan dosages range from 1 to 100 mg/kg/day, eplerenone dosages range from 1 to 300 mg/kg/day, and HCTZ dosages range from 1 to 75 mg/kg/day.

In addition to the cardiovascular parameters, weekly determinations of body weight also are recorded in all rats. Treatments are administered in the drinking water, via daily oral gavage, mixed in food or in osmotic minipumps as stated above. If given in drinking water, water consumption is measured five times per week. Valsartan, eplerenone, and HCTZ doses for individual rats are then calculated based on water or food consumption for each rat, the concentration of drug substance in the drinking water or proportion of drug substance in the food, and individual body weights. All drug solutions in the drinking water or food admixtures with drug are made up fresh every three to four days. In most situations, the daily dose will not exceed 300 mg/kg/day when administered as the monotherapy. In combination, lower dosages of each agent are used and correspondingly, valsartan is given in the range of 1 to 30 mg/kg/day, eplerenone is give in dosages ranging from 1 to 100 mg/kg/day and HCTZ is give in dosages below 50 mg/kg/day.

Upon completion of the chronic studies, the rats are anesthetized, blood samples obtained for biochemical analysis and the heart rapidly removed. After separation and removal of the atrial appendages, left ventricle and left plus right ventricle (total) are weighed and recorded. Left ventricular and total ventricular mass are then normalized to body weight and reported.

Vascular function and structure are evaluated after treatment to assess the beneficial effects of the combination. SHRsp are studied according to the methods described for spontaneously hypertensive rats by Intengan H D, Thibault G, Li J S, Schiffrin E L, Circulation 1999, 100 (22): 2267-2275. Similarly, the methodology for assessing vascular properties in SHHF is as described for the Dahl salt-sentive rat in Intengan H D, and Schiffrin, E L, J. Hypertension, 1998, 16(12 Part 2): 1907-912. Assessment of vascular compliance and distensibility following treatment with the combination regimen is performed according to the methods described by Ceiler D L, Nelissen-Vrancken H J, De Mey J G, Smits J F, J Cardiovasc Pharmacol 1998, 31 (4):630-7. Amelioration of cardiac, renal, and cerebral injury secondary to hypertension is assessed after treatment with the combination regimen in salt-supplemented SHRsp according to the methods described by Nagura J, Yamamoto M, Hui C, Yasuda S, Hachisu M, Konno F, Clin Exp Pharmacol Physiol 1996, 23(3):229-35. Propensity of the combination therapy to elicit postural or orthostatic hypotension is assessed in SHRsp by the methods described by Nabata H, Aono J, Ishizuka N, Sakai K, Arch Int Pharmacodyn Ther 1985, 277(1):104-18.

Valsartan is supplied in the form of suitable dosage unit form, for example, a capsule or tablet, and comprising a therapeutically effective amount, e.g. from about 20 to about 320 or 640 mg, of valsartan which may be applied to patients. The application of the active ingredient may occur up to three times a day, starting e.g. with a daily dose of 20 mg or 40 mg of valsartan, increasing via 80 mg daily and further to 160 mg daily up to 320 or 640 mg daily. Preferably, valsartan is applied once a day or twice a day in heart failure patients with a dose of 80 mg or 160 mg, respectively, each. Corresponding doses may be taken, for example, in the morning, at mid-day or in the evening. Preferred is q.d. or b.i.d. administration in heart failure.

In case of eplerenone, preferred dosage unit forms are, for example, tablets or capsules comprising e.g. from about 10 mg to about 1000 mg, preferably an amount of about 10 mg to about 100 mg, more preferably from about 25 mg to about 100 mg, and still more preferably from about 25 mg to about 50 mg. Dosage unit forms of the pharmaceutical compositions may typically contain, for example, 10, 20, 25, 37.5, 50, 75, 100, 125, 150 mg of eplerenone. Preferred dosage unit forms contain about 25, 50 or 100 mg of eplerenone. The dosage unit form may be selected to accommodate the desired frequency of administration used to achieve the specified daily dosage. The amount of the unit dosage form of the pharmaceutical composition that is administered and the dosage regimen for treating the condition or disorder will depend on a variety of factors, including the age, weight, sex and medical condition of the subject, the severity of the condition or disorder, the route and frequency of administration, and thus may vary widely.

For the treatment of heart failure, the pharmaceutical composition preferably provides a daily dosage of eplerenone in the amount of about 25 mg to about 200 mg, more preferably about 25 mg to about 75 mg, and still more preferably about 50 mg. A daily dose of about 0.33 to 2.67 mg/kg body weight (based upon an average body weight of about 75 kg), preferably between about 0.33 and about 1.00 mg/kg body weight and most preferably 0.67 mg/kg body weight, may be appropriate. The daily dose can be administered in one to four doses per day, preferably one dose per day.

For the treatment of hypertension, the pharmaceutical composition preferably provides a daily dosage of eplerenone in the amount of about 50 mg to about 300 mg, more preferably about 50 mg to about 150 mg, and still more preferably about 100 mg. A daily dose of about 0.67 to 4.00 mg/kg body weight, preferably between about 0.67 and about 2.00 mg/kg body weight and most preferably about 1.33 mg/kg body weight, may be appropriate. The daily dose can be administered in one to four doses per day, preferably one dose per day.

For the treatment of edema associated with liver insufficiency, the pharmaceutical composition preferably provides a daily dosage of eplerenone in the amount of about 50 mg to about 500 mg, more preferably about 100 mg to 400 about mg, and still more preferably about 300 mg. A daily dose of about 0.67 to 6.67 mg/kg body weight, preferably between about 1.33 and about 5.33 mg/kg body weight and most preferably about 4.00 mg/kg body weight, may be appropriate. The daily dose can be administered in one to four doses per day, preferably one dose per day.

In case of spironolactone, the minimum effective anti-hypertensive dosage in adults is about 50 milligrams (mg) per day; dosages often exceed this, and dosages of 200 to 400 mg/day are common for chronic treatment. Since spironolactone is metabolized and secreted fairly rapidly, typical administration involves pills containing 25 to 100 mg, taken four times daily. spironolactone, which is marketed as an anti-hypertensive and diuretic drug by G. D. Searle (Skokie, III.) under the trademarks “Aldactone” and “Aldactazide.”

In case of HCTZ, preferred dosage unit forms are, for example, tablets or capsules comprising e.g. from about 5 mg to about 200 mg preferably from about 50 mg to about 150 mg, even more preferably from about 25 mg to about 100 mg and even more preferably from about 5 mg to about 25 mg, administered orally once a day.

An example of a preferred combination, comprises an amount of Valsartan between 60 and 180 mg e.g. 80 mg or 160 mg, an amount of eplerenone between 25 and 100 mg e.g. 25 or 50 mg or 100 mg and an amount of HCTZ between 8 and 16 mg e.g. 12.5 mg.

Another example of a preferred combination, comprises an amount of Valsartan between 140 and 180 mg e.g. 160 mg, an amount of eplerenone between 25 and 100 mg e.g. 25 or 50 or 100 mg and an amount of HCTZ between 8 and 16 mg e.g. 12.5 mg.

Another example of a preferred combination comprises an amount of Valsartan between 140 and 180 mg e.g. 160 mg, an amount of eplerenone between 25 and 100 mg e.g. 25 or 50 mg or 100 mg, and an amount of HCTZ between 20 and 30 mg e.g. 25 mg. In all the herein described preferred combinations, vasartan is either present or not.

In a further preferred embodiment, the ARB e.g. valsartan is not present in the herein described preferred combinations.

An example of such a preferred combination, comprises an amount of eplerenone between 25 and 100 mg e.g. 25 or 50 or 100 mg and an amount of HCTZ between 8 and 16 mg e.g. 12.5 mg.

An example of such a preferred combination, comprises an amount of eplerenone between 25 and 100 mg e.g. 25 or 50 mg or 100 mg, and an amount of HCTZ between 20 and 30 mg e.g. 25 mg.

Most preferred combinations (fixed combination or combined preparations) are summarized below;

| | | |
|---|---|---|
| eplerenone | HCTZ | valsartan |

| | | | | | | |
|---|---|---|---|---|---|---|
| Combination 1 | 25 | mg | 12.5 | mg | 40 | mg |
| Combination 2 | 50 | mg | 12.5 | mg | 40 | mg |
| Combination 3 | 100 | mg | 12.5 | mg | 40 | mg |
| Combination 4 | 25 | mg | 25 | mg | 40 | mg |
| Combination 5 | 50 | mg | 25 | mg | 40 | mg |
| Combination 6 | 100 | mg | 25 | mg | 40 | mg |
| Combination 7 | 25 | mg | 12.5 | mg | 80 | mg |
| Combination 8 | 50 | mg | 12.5 | mg | 80 | mg |
| Combination 9 | 100 | mg | 12.5 | mg | 80 | mg |
| Combination 10 | 25 | mg | 12.5 | mg | 160 | mg |
| Combination 11 | 50 | mg | 12.5 | mg | 160 | mg |
| Combination 12 | 100 | mg | 12.5 | mg | 160 | mg |
| Combination 13 | 25 | mg | 25 | mg | 80 | mg |
| Combination 14 | 50 | mg | 25 | mg | 80 | mg |
| Combination 15 | 100 | mg | 25 | mg | 80 | mg |
| Combination 16 | 25 | mg | 25 | mg | 160 | mg |
| Combination 17 | 50 | mg | 25 | mg | 160 | mg |
| Combination 18 | 100 | mg | 25 | mg | 160 | mg |

| | | | | | |
|---|---|---|---|---|---|
| Combination 19 | 25 | mg | 12.5 | mg | — |
| Combination 20 | 50 | mg | 12.5 | mg | — |
| Combination 21 | 100 | mg | 12.5 | mg | — |
| Combination 22 | 25 | mg | 25 | mg | — |
| Combination 23 | 50 | mg | 25 | mg | — |
| Combination 24 | 100 | mg | 25 | mg | — |

The combination of, (ii) an aldosterone receptor antagonist, (ii) a diuretic and optionally (iii) an ARB may, according to the present invention be manufactured and administered in free or fixed dose combinations of the respective pharmaceutically active agents. It may be advantageous to begin the treatment with free combinations that allow an easy adjustment of the administered dose of each individual agent. When the ideal dose regimen, which generally is dependent on the specific condition of the individual to be treated, the individuals weight, other medication administered to the individual and the like, is reached, a fixed dose combination may be administered in case where an administration once a day or e.g. twice or three times daily is possible and a sufficient control of blood pressure is achieved.

Presently it is preferred to combine two of the components (i) to (iii) and administer the third separately at the same or at a different time.

Valsartan is being marketed under the trade name Diovan®. A combination of valsartan and HCTZ is being marketed under the trade name Co-Diovan® and eplerenone is being marketed under the trade name INSPRA®. All of these marketed products may be utilized in as such for combination therapy according to the present invention. HCTZ is being marketed e.g. under the trade name Aldoril®, Oretic®, Esidrex® or Esidrix® and many other trade names known by the skilled person.

INSPRA for oral administration contains 25 mg, 50 mg, or 100 mg of eplerenone and the following inactive ingredients: lactose, microcrystalline cellulose, croscarmellose sodium, hydroxypropyl methylcellulose, sodium lauryl sulfate, talc, magnesium stearate, titanium dioxide, polyethylene glycol, polysorbate 80, and iron oxide yellow and iron oxide red (25 mg tablet) and iron oxide red (50 and 100 mg tablets).

The following examples illustrate the invention described above and are not intended to restrict the scope of this invention in any way.

### FORMULATION EXAMPLE 1

Composition and batch quantities for Diovan ® tablets

| | COMPOSITION PER UNIT (mg) | | | | QUANTITY PER BATCH¹ (kg) | | | |
|---|---|---|---|---|---|---|---|---|
| Components | 40 mg | 80 mg | 160 mg | 320 mg | 40 mg | 80 mg | 160 mg | 320 mg |
| Granulation | | | | | | | | |
| Diovan Drug Substance | 40.000 | 80.000 | 160.000 | 320.000 | 144.000 | 144.000 | 144.000 | 144.000 |
| Microcrystalline | 27.000 | 54.000 | 108.000 | 216.000 | 97.200 | 97.200 | 97.200 | 97.200 |
| Cellulose(NF, Ph. Eur.) | | | | | | | | |
| Avicel PH102 | | | | | | | | |
| Crospovidone | 7.500 | 15.000 | 30.000 | 60.000 | 27.000 | 27.000 | 27.000 | 27.000 |
| (NF, Ph. Eur.) | | | | | | | | |
| Colloidal Anhydrous Silica | 0.750 | 1.500 | 3.000 | 6.000 | 2.700 | 2.700 | 2.700 | 2.700 |
| (Ph. Eur.)/Colloidal silicon | | | | | | | | |
| Dioxide (NF)/Aerosil 200 | | | | | | | | |
| Magnesium Stearate | 1.500 | 3.000 | 6.000 | 12.000 | 5.400 | 5.400 | 5.400 | 5.400 |
| (NF, Ph. Eur.) | | | | | | | | |
| Blending | | | | | | | | |
| Magnesium Stearate | 0.750 | 1.500 | 3.000 | 6.000 | 2.700 | 2.700 | 2.700 | 2.700 |
| (NF, Ph. Eur.) | | | | | | | | |
| Coating | | | | | | | | |
| DIOLACK Gelb F32892 | 2.800 | | | | 11.090² | | | |
| DIOLACK Blassrot F34899 | | 6.000 | | | | 12.420³ | | |
| DIOLACK Hellbraun F33172 | | | 9.000 | | | | 9.720⁴ | |
| DIOLACK Braun F16711 | | | | 16.000 | | | | 8.640⁴ |
| Purified Water | | | | | 62.843 | 70.380 | 55.080 | 48.960 |
| Total Tablet/Batch Weight | 80.300 | 161.000 | 319.000 | 636.000 | 289.080 | 289.800 | 287.100 | 286.200 |

¹A total of 2 subdivisions of granulation per batch
²A 10% excess of coating solution was manufactured to account for loss during coating.
³A 15% excess of coating solution was manufactured to account for loss during coating.
⁴A 20% excess of coating solution was manufactured to account for loss during coating.

Composition of Diolack

| | | | | Iron Oxide | Iron Oxide | Iron Oxide | |
|---|---|---|---|---|---|---|---|
| | | | Titanium | (Red) | (Yellow) | (Brown) | Iron Oxide |
| | HPMC | | Dioxide | Ph. Fr./NF/ | Ph. Fr./NF/ | Mixture of iron | (Black) |
| | USP/Ph. Eur | PEG 8000 | (White) | E172/CFR/ | E172/CFR/ | oxide red & | E172/CFR/ |
| DIOLACK | (603) | USP/Ph. Eur. | USP/Ph. Eur | CI 77491 | CI 77492 | black | CI 77499 |
| Gelb | 80.00% | 4.00% | 13.48% | 0.01% | 2.50% | — | 0.01% |
| F32892 | | | | | | | |
| Blassrot | 80.00% | 4.00% | 15.50% | 0.40% | 0.10% | — | — |
| F34899 | | | | | | | |
| Hellbraun | 80.00% | 4.00% | 9.34% | 0.25% | 6.40% | — | 0.01% |
| F33172 | | | | | | | |
| Braun | 80.00% | 4.00% | 14.00% | 0.50% | 0.50% | 0.50% | 0.50% |
| F16711 | | | | | | | |

A mixture of Diovan drug substance, microcrystalline cellulose, crospovidone, part of the colloidal anhydrous silica/colloidal silicon dioxide/Aerosil 200, silicon dioxide and magnesium stearate is premixed in a diffusion mixer and then sieved through a screening mill. The resulting mixture is again pre-mixed in a diffusion mixer, compacted in a roller compacter and then sieved through a screening mill. To the resulting mixture, the rest of the colloidal anhydrous silica/colloidal silicon dioxide/Aerosil 200 are added and the final blend is made in a diffusion mixer. The whole mixture is compressed in a rotary tabletting machine and the tablets are coated with a film by using the appropriate composition of Diolack in a perforated pan.

### FORMULATION EXAMPLE 2

Composition and quantities for Co-Diovan ® tablets

| | COMPOSITION PER | COMPOSITION PER | COMPOSITION PER |
|---|---|---|---|
| Components | UNIT (mg) | UNIT (mg) | UNIT (mg) |
| Granulation | | | |
| Diovan Drug Substance | 80.000 | 160.000 | 160.00 |
| Esidrex Drug Substance | 12.500 | 12.500 | 25.00 |
| (micro) | | | |
| Microcrystalline Cellulose | 31.500 | 75.500 | 63.00 |
| (NF, Ph. Eur.)/Avicel PH 102 | | | |
| Crospovidone (NF, Ph. Eur.) | 20.000 | 40.000 | 40.00 |
| Colloidal Anhydrous Silica | 1.500 | 3.00 | 3.00 |
| (Ph. Eur.)/Colloidal Silicon | | | |
| Dioxide (NF)/Aerosil 200 | | | |
| Magnesium Stearate | 3.000 | 6.000 | 6.00 |
| (NF, Ph. Eur.) | | | |
| Blending | | | |
| Magnesium Stearate, | 1.500 | 3.000 | 3.00 |
| NF, Ph. Eur. | | | |
| Coating | | | |
| Opadry Black OOF17713 | — | — | 0.096 |
| Opadry Red OOF15613 | — | — | 0.762 |
| Opadry Yellow OOF12951 | — | — | 3.808 |
| Opadry White OOF18296 | — | — | 5.334 |
| Hydroxy propyl | 2.76 | 5.510 | — |
| Methylcellulose | | | |
| Iron Oxide Yellow | 0.025 | — | — |
| Iron Oxide Red | 0.025 | 0.750 | — |
| Polyethylene Glycol 8000 | 0.50 | 1.000 | — |
| Talc | 2.000 | 3.990 | — |
| Titanium Dioxide | 0.70 | 0.750 | — |
| Total Tablet/Batch Weight | 156.000 | 312.000 | 310.00 |

Preferred combinations comprise Co-Diovan 80 (Diovan)/12.5(Esidrex) or 160/12.5 with either 25 or 50 mg of eplerenone e.g. INSPRA®.

Composition of Opadry

| | | | | | Iron Oxide | Iron Oxide | |
|---|---|---|---|---|---|---|---|
| | | | | Titanium | (Red) | (Yellow) | IronOxide |
| | HPMC | | | Dioxide | Ph. Fr./NF/ | Ph. Fr./NF/ | (Black) |
| | USP/Ph. Eur | PEG 4000 | Talc | USP/Ph. Eur | E172/CFR/ | E172/CFR/ | E172/CFR/ |
| OPADRY | (603) | USP/Ph. Eur. | USP/Ph. Eur | (White) | CI 77491 | CI 77492 | CI 77499 |
| Opadry White | 71.4% | 7.15% | 7.15% | 14.3% | — | — | — |
| OOF18296* | | | | | | | |
| Opadry Red | 71.4% | 7.15% | 7.15% | — | 14.3% | — | — |
| OOF15613* | | | | | | | |
| Opadry Red | 71.4% | 7.15% | 7.15% | — | — | 14.3% | — |
| OOF15613* | | | | | | | |
| Opadry Black | 71.4% | 7.15% | 7.15% | — | — | — | 14.3% |
| OOF17713* | | | | | | | |

A mixture of Diovan drug substance, Esidrex drug substance (micro), microcrystalline cellulose, crospovidone, colloidal anhydrous silica/Aerosil 200 and part of the magnesium stearate is premixed in a diffusion mixer and then sieve through a screening mill. The resulting mixture is again pre-mixed in a diffusion mixer, compacted in a roller compacter and then sieved through a screening mill. The final blend is made in a diffusion mixer under addition of the remaining part of the magnesium stearate, which is hand screened before. The whole mixture is compressed in a rotary tabletting machine and the tablets are coated with a film by using the appropriate composition of Opadry in a perforated pan.

Composition and quantities for a combination
of valsartan and eplerenone

| | COMPOSITION PER | COMPOSITION |
|---|---|---|
| Components | UNIT (mg) | (%) |
| Diovan Drug Substance | 80.00 | 39.22 |
| INSPRA ®. (i.e. eplerenone) | 25 | 12.25 |
| Drug Substance | | |
| Avicel 102 (I) | 54.00 | 26.47 |
| Avicel 102 (II) | 20.00 | 9.80 |
| Crospovidone (I) | 15.00 | 7.35 |
| Crospovidone (II) | 4.0 | 1.96 |
| Cab-O-Sil | 1.50 | 0.74 |
| Magnesium Stearate (I) | 3.00 | 1.47 |
| Magnesium Stearate (II) | 1.50 | 0.74 |
| | 204.00 | 100.00 |

### FORMULATION EXAMPLE 3

The tablet is manufactured e.g essentially as described in Formulation Example 1.

1. A combination comprising
(i) an aldosterone receptor antagonist or a pharmaceutically acceptable salt thereof,
(ii) a diuretic or a pharmaceutically acceptable salt thereof, and optionally
(iii) an angiotensin receptor blocker (ARB) or a pharmaceutically acceptable salt thereof.

2. A combination according to claim 1, wherein
(i) the aldosterone receptor antagonist is selected from the group consisting of spironolactone or eplerenone;
(ii) the diuretic is selected from the group consisting of bumetanide, ethacrynic acid, furosemide, ethynacrylic acid, mersalyl with theophylline, mercaptomerin sodium, merethoxylline procaine, torsemide, amiloride, spironolactone, triamterene, chlorothalidone, chlorothiazide, quinethazone,hydrochlorothiazide (HCTZ), hydroflumethiazide, methylchlorothiazide, metolazone, and dichlorphenamide; and
(iii) the angiotensin receptor blocker (ARB) is selected from the group consisting of candesartan, eprosartan, irbesartan, losartan, olmesartan, saprisartan, tasosartan, telmisartan, valsartan, E-4177, SC-52458, and ZD8731, or a pharmaceutically acceptable salts thereof.

3. A combination according to claim 2, wherein (i) the aldosterone receptor antagonist is eplerenone; (ii) the diuretic is hydrochlorothiazide; and (iii) the angiotensin receptor blocker (ARB) is valsartan.

4. A combination according to claim 3, wherein valsartan is contained in an amount from about 20 to about 640 mg, eplerenone is contained in an amount from about 10 mg to about 1000 mg, and hydrochlorothiazide is contained in an amount from about 5 mg to about 200 mg.

5. A combination according to claim 4, wherein valsartan is contained in an amount from about 40 to about 320 mg, eplerenone is contained in an amount from about 10 mg to about 100 mg, and hydrochlorothiazide is contained in an amount from about 5 mg to about 25 mg.

6. Combination according to claim 1 in the form of a combined preparation or a pharmaceutical composition.

7. A kit of parts comprising
(i) a pharmaceutical composition of an aldosterone receptor antagonist,
(ii) a pharmaceutical composition of a diuretic, and optionally
(iii) a pharmaceutical composition of an angiotensin receptor blocker (ARB) in the form of two or three separate units of the components (i) to (ii) or (i) to (iii).

8. A kit of parts according to claim 7, wherein (i) the aldosterone receptor antagonist is selected from the group consisting of spironolactone or eplerenone;
(ii) the diuretic is selected from the group consisting of bumetanide, ethacrynic acid, furosemide, ethynacrylic acid, mersalyl with theophylline, mercaptomerin sodium, merethoxylline procaine, torsemide, amiloride, spironolactone, triamterene, chlorothalidone, chlorothiazide, quinethazone,hydrochlorothiazide (HCTZ), hydroflumethiazide, methylchlorothiazide, metolazone, and dichlorphenamide; and
(iii) the angiotensin receptor blocker (ARB) is selected from the group consisting of candesartan, eprosartan, irbesartan, losartan, olmesartan, saprisartan, tasosartan, telmisartan, valsartan, E-4177, SC-52458, and ZD8731, or a pharmaceutically acceptable salts thereof

9. A kit of parts according to claim 8, wherein (i) the aldosterone receptor antagonist is eplerenone; (ii) the diuretic is hydrochlorothiazide; and (iii) the angiotensin receptor blocker (ARB) is valsartan.

10. A kit of parts according to claim 9, wherein valsartan is contained in an amount from about 20 to about 640 mg, eplerenone is contained in an amount from about 10 mg to about 1000 mg, and hydrochlorothiazide is contained in an amount from about 5 mg to about 200 mg.

11. A kit of parts according to claim 10, wherein valsartan is contained in an amount from about 40 to about 320 mg, eplerenone is contained in an amount from about 10 mg to about 100 mg, and hydrochlorothiazide is contained in an amount from about 5 mg to about 25 mg.

12. A method of treatment or prevention of a condition or disease selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke, comprising administering a therapeutically effective amount of combination of
(i) an aldosterone receptor antagonist or-a pharmaceutically acceptable salt thereof,
(ii) a diuretic or a pharmaceutically acceptable salt thereof, and optionally
(iii) an angiotensin receptor blocker (ARB) or, where appropriate, a pharmaceutically acceptable salt thereof and a pharmaceutically acceptable carrier to a mammal in need of such treatment.

13. A commercial package comprising
(i) a pharmaceutical composition of an aldosterone receptor antagonist,
(ii) a pharmaceutical composition of a a diuretic , and optionally
(iii) a pharmaceutical composition of a an angiotensin receptor blocker (ARB) in the form of two or three separate units of the components (i) to (ii) or (i) to (iii), together with instructions for simultaneous, separate or sequential use thereof for the treatment or prevention of a condition or disease selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke.

14. A commercial package according to claim 13, wherein wherein (i) the aldosterone receptor antagonist is eplerenone; (ii) the diuretic is hydrochlorothiazide; and (iii) the angiotensin receptor blocker (ARB) is valsartan.

15. A commercial package according to claim 14, wherein the angiotensin receptor blocker (ARB) (iii) and the diuretic (ii) are present in the form of Co-DIOVAN®.

16. The use of a combination according to claim 1, for the manufacture of a medicament for the treatment or prevention of a condition or disease selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non-diabetic), heart failure, angina pectoris, diabetes, secondary aldosteronism, primary and secondary pulmonary hypertension, renal failure conditions, such as diabetic nephropathy, glomerulonephrits, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke.

17. The use of a combination according to claim 1, for the treatment or prevention of a condition or disease selected from the group consisting of hypertension, heart failure such as (acute and chronic) congestive heart failure, left ventricular dysfunction and hypertrophic cardiomyopathy, diabetic cardiac myopathy, supraventricular and ventricular arrhythmias, atrial fibrillation, atrial flutter, detrimental vascular remodeling, myocardial infarction and its sequelae, atherosclerosis, angina (whether unstable or stable), renal insufficiency (diabetic and non- diabetic), heart failure, angina pectoris, diabetes, secondary aldosteronism, primary and secondary pulmonary hypertension, renal failure conditions, such as diabetic nephropathy, glomerulonephritis, scleroderma, glomerular sclerosis, proteinuria of primary renal disease, and also renal vascular hypertension, diabetic retinopathy, the management of other vascular disorders, such as migraine, peripheral vascular disease, Raynaud's disease, luminal hyperplasia, cognitive dysfunction (such as Alzheimer's), glaucoma and stroke.
